(19) United States
(12) Patent Application Publication
(10) Pub. No.: US 2025/0302927 A1
(43) Pub. Date: Oct. 2, 2025

(54) MODIFIED FACTOR XA POLYPEPTIDES AND METHODS OF USE

(71) Applicant: The United States of America, as represented by the Secretary, Dept. of Health and Human Services, Silver Spring, MD (US)

(72) Inventors: Zuben E. Sauna, Rockville, MD (US); Wojciech Jankowski, Potomac, MD (US); Nancy E. Hernandez, Beltsville, MD (US)

(73) Assignee: The United States of America, as represented by the Secretary, Dept. of Health and Human Services, Silver Spring, MD (US)

(21) Appl. No.: 18/863,242

(22) PCT Filed: May 5, 2023
(86) PCT No.: PCT/US2023/066676

§ 371 Date: Nov. 5, 2024

Related U.S. Application Data
(60) Provisional application No. 63/339,230, filed on May 6, 2022.

(51) Int. Cl. A61K 38/48; A61P 7/04; C12N 9/64

(52) U.S. Cl. CPC A61K 38/4846; A61P 7/04; C12N 9/6432; C12Y 304/21006

(57) ABSTRACT

Modified Factor Xa polypeptides having functional activity and reduced affinity for apixaban are provided. Nucleic acids encoding the modified Factor Xa polypeptides and vectors and host cells including the nucleic acids are also provided. Methods of reducing or inhibiting bleeding, such as in a subject being treated with a direct oral anticoagulant are provided.

### CROSS REFERENCE TO RELATED APPLICATIONS

This application claims priority to U.S. Provisional Application No. 63/339,230, filed May 6, 2022, which is incorporated by reference in its entirety.

### FIELD

This disclosure relates to modified Factor Xa polypeptides and methods of their use, particularly for reducing or inhibiting the effects of direct oral anticoagulants that inhibit Factor Xa.

### BACKGROUND

Direct oral anticoagulants (DOACs) that directly inhibit Factor Xa (FXa) have revolutionized the prevention of systemic embolization and stroke. Five DOACs have been approved. Of these, one (dabigatran) inhibits Factor IIa, while four (rivaroxaban, apixaban, edoxaban, and betrixaban) are FXa inhibitors. The DOACs are widely prescribed for the prevention of systemic embolization and stroke, the treatment of nonvalvular atrial fibrillation, and venous thromboembolism. While FXa inhibitors have shown a favorable benefit-risk profile in the prevention and/or treatment of thrombotic events, these agents are also associated with acute major bleeding. More than 100,000 DOAC-related major bleeding cases occur each year in the United States and European Union and are difficult to treat. The only currently approved specific reversal agent for FXa inhibitors, andexanet alfa (Andexxa®), is a bioengineered inactive variant of human FXa. Andexxa® has been demonstrated to bind and sequester FXa inhibitors. Andexxa® and FXa have comparable affinities for DOACs, consequently, the recommended low dose initial intravenous (IV) bolus is 400 mg at a target rate of 30 mg/min followed by IV infusion at 4 mg/min for up to 120 minutes, and the recommended high dose initial IV bolus is 800 mg at a target rate of 30 mg/min followed by IV infusion at 8 mg/min for up to 120 min. Risks associated with Andexxa® include arterial and venous thrombosis, myocardial infarction, ischemic stroke, cardiac arrest, and sudden death.

### SUMMARY

A safe and effective DOAC reversal agent remains an unmet need. The design of functional Factor Xa analogs for which DOACs have low affinity offer an alternative reversal strategy. Disclosed herein are functional Factor Xa analogs to which apixaban (the most widely prescribed Factor Xa inhibitor) has low affinity.

Provided herein are modified Factor Xa polypeptide comprising one or more amino acid substitutions in Factor Xa subsite S1, S4, or both, and having enzymatic activity of at least 300% of unmodified Factor Xa in the presence of apixaban, IC₅₀ value for apixaban of at least 50-fold higher than unmodified Factor Xa, or both. In some aspects, the unmodified Factor Xa includes or consists of the amino acid sequence of SEQ ID NO: 1. In additional aspects, the modified Factor Xa polypeptide does not include the signal peptide and propeptide of Factor Xa (e.g., does not include amino acids 1-40 of SEQ ID NO: 1).

In specific aspects provided herein, the modified Factor Xa polypeptide includes one or more amino acid substitutions selected from W439A, 312-317NRFTKE→STYVPG, G450A, G440A, C415A+C443A, 312-317NRFTKE→KNYQRD, and W439A+312-317NRFTKE→STYVPG, wherein the amino acid numbering corresponds to SEQ ID NO: 1. In some examples, the modified Factor Xa polypeptide includes one of W439A, 312-317NRFTKE→STYVPG, G450A, G440A, C415A+C443A, 312-317NRFTKE→KNYQRD, and W439A+312-317NRFTKE→STYVPG and has an amino acid sequence with at least 95% sequence identity to any one of SEQ ID NOs: 2, 4, 6, 8, 10, 12, and 14, or includes or consists of the amino acid sequence of any one of SEQ ID NOs: 2, 4, 6, 8, 10, 12, and 14. In other aspects, the modified Factor Xa polypeptide includes one of W439A, 312-317NRFTKE→STYVPG, G450A, G440A, C415A+C443A, 312-317NRFTKE→KNYQRD, and W439A+312-317NRFTKE→STYVPG and has an amino acid sequence with at least 95% sequence identity to amino acids 3-450 of any one of SEQ ID NOs: 2, 4, 6, 8, 10, 12, and 14, or includes or consists of the amino acid sequence of amino acids 3-450 of any one of SEQ ID NOs: 2, 4, 6, 8, 10, 12, and 14.

Also provided are nucleic acids encoding the disclosed modified Factor Xa polypeptides. In some aspects, the nucleic acid encodes a polypeptide with one of W439A, 312-317NRFTKE→STYVPG, G450A, G440A, C415A+C443A, 312-317NRFTKE→KNYQRD, and W439A+312-317NRFTKE→STYVPG and has a nucleotide sequence with at least 95% sequence identity to any one of SEQ ID NOs: 3, 5, 7, 9, 11, 13, and 15, or includes or consists of the nucleotide sequence of any one of SEQ ID NOs: 3, 5, 7, 9, 11, 13, and 15. In other aspects, the nucleic acid encodes one of W439A, 312-317NRFTKE→STYVPG, G450A, G440A, C415A+C443A, 312-317NRFTKE→KNYQRD, and W439A+312-317NRFTKE→STYVPG and has a nucleotide sequence with at least 95% sequence identity to nucleotides 7-1350 of any one of SEQ ID NOs: 3, 5, 7, 9, 11, 13, and 15, or includes or consists of nucleotides 7-1350 of any one of SEQ ID NOs: 3, 5, 7, 9, 11, 13, and 15. Also provided are vectors including a nucleic acid encoding a disclosed modified Factor Xa polypeptide and host cells including the nucleic acids or vectors.

In some aspects, a composition including a disclosed modified Factor Xa polypeptide and a pharmaceutically acceptable carrier is provided.

Methods of treating or inhibiting bleeding in a subject receiving direct oral anticoagulant therapy are also provided. The methods include administering to the subject an effective amount of a disclosed modified Factor Xa polypeptide, thereby treating or inhibiting the bleeding. In some aspects, the subject is receiving the DOAC apixaban, rivaroxaban, edoxaban, or betrixaban. In one example the subject is receiving apixaban. In some examples, the modified Factor Xa polypeptide or composition is administered to the subject intravenously. In some examples, the modified Factor Xa polypeptide or composition is administered to the subject at a dose of about 0.1-10 mg/kg. Treatment with the modified Factor Xa polypeptide may reduce bleeding time by at least 10% compared to a control, reduce blood loss by at least 10% compared to a control, or both.

The foregoing and other features of the disclosure will become more apparent from the following detailed description of several aspects, which proceeds with reference to the accompanying figures.

### BRIEF DESCRIPTION OF THE DRAWINGS

FIGS. 1A-1H show design and functional characterization of low affinity FXa variants. FIG. 1A illustrates the computational strategy using RosettaDesign and homology insertion. FIG. 1B shows models of FXa variants 61 (upper panel) and 70 (lower panel) showing mutations that affect apixaban binding. Variants 61 and 70 and apixaban are shown as stick models. FIG. 1C shows location of variants on the FXa heavy chain region (catalytic domain). Variants 69 and 70 are located on the same region between H311 and T318. FIG. 1D shows location of variants represented as a “surface” with apixaban occupying the binding pocket. FIG. 1E shows the S1 and S4 subsites of the binding pocket. FIG. 1F is a graph showing the rate of chromogenic substrate cleavage in pure buffer systems by FXa variants at different concentrations. FXa HTI (commercially available plasma derived FXa) was used to evaluate in-house made FXa (FXa WJ) to determine if activities are comparable. FIG. 1G is a graph showing the rate of chromogenic substrate cleavage by 0.5 μg of FXa variants in the absence (V0) or presence (Vi) of increasing apixaban concentrations. Values for IC₅₀±SD were obtained from fitted curves. FIG. 1H shows ¹H NMR stacked spectra of Factor VII (FVII) with stoichiometric amounts of Apixaban (top), negative control sample; Apixaban (middle), reference spectrum; and rFXa:Apixaban in stoichiometric proportions (bottom), test sample. The top spectrum is the result of superposition of ligand and protein signals with no alteration. The reference NMR spectrum (middle) contains only sharp signals, as expected for an NMR spectrum of a small molecule. However, even though there is a small molecule in the sample, the test sample ¹H NMR spectrum (bottom) shows only broadened NMR signals, indicative of strong Apixaban binding to rFXa.

FIGS. 2A and 2B show the rate of chromogenic substrate cleavage by different amounts of FXa variants in the absence (V0) or presence (Vi) of increasing apixaban concentrations. Values for IC₅₀±SD were obtained from fitted curves. Variants are as described in Tables 1 and 2.

FIGS. 3A-3E show effect of variants 61 and 70 on in vitro and in vivo procoagulant potential in the presence of apixaban (Apix or A). FIG. 3A shows effects of FXa variants on thrombin generation in plasma spiked with apixaban. Thrombin peak height (TPH) values obtained in titration of indicated concentrations of FXa variants in normal pooled plasma spiked with 1 μM of apixaban. Experimental design (FIG. 3B) and readout parameters of mice tail-clipping model: bleeding time (FIG. 3C), blood loss (FIG. 3D) and bleeding profiles (FIG. 3E).

FIGS. 4A-4D show effect of variants 61 and 70 and andexanet alfa (Andx) on in vitro and in vivo procoagulant potential in the presence of apixaban (APX). FIG. 4A shows the experimental design. FIG. 4B shows bleeding time, FIG. 4C shows blood loss, and FIG. 4D shows bleeding profiles for the indicated treatments.

FIG. 5 shows tissue factor pathway inhibitor (TFPI) activity in response to FXa, 4 nM variants 61 or 70, and 100 nM andexanet alfa (Andx).

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an exemplary Factor X preproprotein amino acid sequence (signal peptide, amino acids 1-31; propeptide, amino acids 32-40; Gla domain, amino acids 41-85; EGF-like domain 1, amino acids 86-122; EGF-like domain 2, amino acids 125-165; activation peptide, amino acids 179-234; catalytic domain, amino acids 235-488):

MGRPLHLVLLSASLAGLLLLGESLFIRREQANNILARVTRANSFLEEMKKGHLERECMEETCSYEEA
REVFEDSDKTNEFWNKYKDGDQCETSPCQNQGKCKDGLGEYTCTCLEGFEGKNCELFTRKLCSLDNG
DCDQFCHEEQNSVVCSCARGYTLADNGKACIPTGPYPCGKQTLERRKRSVAQATSSSGEAPDSITWK
PYDAADLDPTENPFDLLDENQTQPERGDNNLTRIVGGQECKDGECPWQALLINEENEGFCGGTILSE
FYILTAAHCLYQAKRFKVRVGDRNTEQEEGGEAVHEVEVVIKHNRFTKETYDFDIAVLRLKTPITER
MNVAPACLPERDWAESTLMTQKTGIVSGFGRTHEKGRQSTRLKMLEVPYVDRNSCKLSSSFIITQNM
FCAGYDTKQEDACQGDSGGPHVTRFKDTYFVTGIVSWGEGCARKGKYGIYTKVTAFLKWIDRSMKTR
GLPKAKSHAPEVITSSPLK
SEQ ID NO: 2 is the amino acid sequence of FXa variant 61:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKRFKVRVGDRNTEQEEGGEAVHEVE
VVIKHNRFTKETYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSAG
EGCARKGKYGIYTKVTAFLKWIDRSMKTRGLPKAKSHAPEVITSSPLK
SEQ ID NO: 3 is a nucleic acid sequence encoding FXa variant 61:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACAACCGGTTCACAAAGGAGACCTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCTACTTCGTGACAGGCATCGTCAGCGCTGGA
GAGGGCTGTGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 4 is the amino acid sequence of FXa variant 70:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKRFKVRVGDRNTEQEEGGEAVHEVE
VVIKHSTYVPGTYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSWG
EGCARKGKYGIYTKVTAFLKWIDRSMKTRGLPKAKSHAPEVITSSPLK
SEQ ID NO: 5 is a nucleic acid sequence encoding FXa variant 70:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACTCAACATACGTTCCGGGAACGTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCTACTTCGTGACAGGCATCGTCAGCTGGGGA
GAGGGCTGTGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 6 is the amino acid sequence of FXa variant 63:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKRFKVRVGDRNTEQEEGGEAVHEVE
VVIKHNRFTKETYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSWG
EGCARKGKYAIYTKVTAFLKWIDRSMKTRGLPKAKSHAPEVITSSPLK
SEQ ID NO: 7 is a nucleic acid sequence encoding FXa variant 63:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACAACCGGITCACAAAGGAGACCTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCTACTTCGTGACAGGCATCGTCAGCTGGGGA
GAGGGCTGTGCCCGTAAGGGGAAGTACGCAATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 8 is the amino acid sequence of FXa variant 65:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKRFKVRVGDRNTEQEEGGEAVHEVE
VVIKHNRFTKETYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSWA
EGCARKGKYGIYTKVTAFLKWIDRSMKTRGLPKAKSHAPEVITSSPLK
SEQ ID NO: 9 is a nucleic acid sequence encoding FXa variant 65:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACAACCGGTTCACAAAGGAGACCTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCIACTTCGTGACAGGCATCGTCAGCTGGGCG
GAGGGCTGTGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 10 is the amino acid sequence of FXa variant 68:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDFNQTQPERGDNNLTRIVGGQ
ECKDGECPWQALLINEENEGFCGGTILSEFYILTAAHCLYQAKRFKVRVGDRNTEQEEGGEAVHEVE
VVIKHNRFTKETYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDAAQGDSGGPHVTRFKDTYFVTGIVSWG
EGAARKGKYGIYTKVTAFLKWIDRSMKTRGLPKAKSHAPEVITSSPLK
SEQ ID NO: 11 is the nucleic acid sequence encoding FXa variant 68:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACAACCGGTTCACAAAGGAGACCTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCGCGCAGGGGGA
CAGCGGGGGCCCGCACGTCACCCGCTTCAAGGACACCTACTTCGTGACAGGCATCGTCAGCTGGGGA
GAGGGCGCGGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 12 is the amino acid sequence of FXa variant 69:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKREKVRVGDRNTEQEEGGEAVHEVE
VVIKHKNYQPDTYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSWG
EGCARKGKYGIYTKVTAFLKWIDRSMKTRGLPKAKSHAPEVITSSPLK
SEQ ID NO: 13 is the nucleic acid sequence encoding FXa variant 69:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACAAGAATTACCAGCCAGACACTTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCIACTTCGTGACAGGCATCGTCAGCIGGGGA
GAGGGCTGTGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG
SEQ ID NO: 14 is the amino acid sequence of FXa variant 73:
GPANSFLEEMKKGHLERECMEETCSYEEAREVFEDSDKTNEFWNKYKDGDQCETSPCQNQGKCKDGL
GEYTCTCLEGFEGKNCELFTRKLCSLDNGDCDQFCHEEQNSVVCSCARGYTLADNGKACIPTGPYPC
GKQTLERRKRSVAQATSSSGEAPDSITWKPYDAADLDPTENPFDLLDENQTQPERGDNNLTRIVGGQ
ECKDGECPWQALLINEENEGFCGGTILSEFYILTAAHCLYQAKRFKVRVGDRNTEQEEGGEAVHEVE
VVIKHSTYVPGTYDFDIAVLRLKTPITFRMNVAPACLPERDWAESTLMTQKTGIVSGFGRTHEKGRQ
STRLKMLEVPYVDRNSCKLSSSFIITQNMFCAGYDTKQEDACQGDSGGPHVTRFKDTYFVTGIVSAG
EGCARKGKYGIYTKVTAFLKWIDRSMKTRGLPKAKSHAPEVITSSPLK
SEQ ID NO: 15 is the nucleic acid sequence encoding FXa variant 73:
GGGCCCGCCAATTCCTTTCTTGAAGAGATGAAGAAAGGACACCTCGAAAGAGAGTGCATGGAAGAGA
CCTGCTCATACGAAGAGGCCCGCGAGGTCTTTGAGGACAGCGACAAGACGAATGAATTCTGGAATAA
ATACAAAGATGGCGACCAGTGTGAGACCAGTCCTTGCCAGAACCAGGGCAAATGTAAAGACGGCCTC
GGGGAATACACCTGCACCTGTTTAGAAGGATTCGAAGGCAAAAACTGTGAATTATTCACACGGAAGC
TCTGCAGCCTGGACAACGGGGACTGTGACCAGTTCTGCCACGAGGAACAGAACTCTGTGGTGTGCTC
CTGCGCCCGCGGGTACACCCTGGCTGACAACGGCAAGGCCTGCATTCCCACAGGGCCCTACCCCTGT
GGGAAACAGACCCTGGAACGCAGGAAGAGGTCAGTGGCCCAGGCCACCAGCAGCAGCGGGGAGGCCC
CTGACAGCATCACATGGAAGCCATATGATGCAGCCGACCTGGACCCCACCGAGAACCCCTTCGACCT
GCTTGACTTCAACCAGACGCAGCCTGAGAGGGGCGACAACAACCTCACCAGGATCGTGGGAGGCCAG
GAATGCAAGGACGGGGAGTGTCCCTGGCAGGCCCTGCTCATCAATGAGGAAAACGAGGGTTTCTGTG
GTGGAACCATTCTGAGCGAGTTCTACATCCTAACGGCAGCCCACTGTCTCTACCAAGCCAAGAGATT
CAAGGTGAGGGTAGGGGACCGGAACACGGAGCAGGAGGAGGGCGGTGAGGCGGTGCACGAGGTGGAG
GTGGTCATCAAGCACTCAACATACGTTCCGGGAACGTATGACTTCGACATCGCCGTGCTCCGGCTCA
AGACCCCCATCACCTTCCGCATGAACGTGGCGCCTGCCTGCCTCCCCGAGCGTGACTGGGCCGAGTC
CACGCTGATGACGCAGAAGACGGGGATTGTGAGCGGCTTCGGGCGCACCCACGAGAAGGGCCGGCAG
TCCACCAGGCTCAAGATGCTGGAGGTGCCCTACGTGGACCGCAACAGCTGCAAGCTGTCCAGCAGCT
TCATCATCACCCAGAACATGTTCTGTGCCGGCTACGACACCAAGCAGGAGGATGCCTGCCAGGGGGA
CAGCGGGGGCCCGCACGTCACCCGCTTCAAGGACACCTACTTCGTGACAGGCATCGTCAGCGCTGGA
GAGGGCTGTGCCCGTAAGGGGAAGTACGGGATCTACACCAAGGTCACCGCCTTCCTCAAGTGGATCG
ACAGGTCCATGAAAACCAGGGGCTTGCCCAAGGCCAAGAGCCATGCCCCGGAGGTCATAACGTCCTC
TCCATTAAAG

### DETAILED DESCRIPTION

Apixaban is the most prescribed DOAC in the US and has proved to be a key medication for the prevention and treatment of thromboembolic disorders. However, safe, and effective reversal agents to control anticoagulant-associated bleeding remain an unmet need. Described herein is rational design of reversal agents for apixaban and other DOACs. The rationale was the design of a FXa variant that bound to apixaban with low affinity but retained sufficient potency to reverse bleeding caused by the use of apixaban. The design of the FXa variants followed a hybrid approach. Rosetta-based computational biology was combined with knowledge of biochemistry and structural biology to iteratively re-design FXa variants. In particular, apixaban binding affinity for two FXa variants that were comprehensively characterized decreased by orders of magnitude. These variants retained sufficient potency to reverse the anti-coagulant effects of apixaban in an in vivo mouse model. The dose of apixaban used in the mouse model tests described herein was comparable to that used in the clinic and the amount of variant FXa used translates to approximately 70 mg for a 70 kg adult. This is considerably lower than the low or high doses of andexanet alfa (Andexxa®), which is the only approved reversal agent that specifically targets FXa inhibitors including apixaban.

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a polypeptide” includes singular or plural polypeptides and can be considered equivalent to the phrase “at least one polypeptide.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided:

Administration: To provide or give a subject an agent, such as a therapeutic agent (e.g. a polypeptide composition), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Direct oral anticoagulant (DOAC): Anticoagulant compounds that directly inhibit thrombin (Factor IIa) or Factor Xa. Currently approved DOACs include Factor IIa inhibitor dabigatran (PRADAXA®) and Factor Xa inhibitors apixaban (ELIQUIS®), rivaroxaban (XARELTO®), and edoxaban (LIXIANA®). DOACs are clinically approved to reduce risk of stroke and systemic embolism in patients with nonvalvular atrial fibrillation, treatment of deep vein thrombosis or pulmonary embolism and to reduce risk of recurrent deep vein thrombosis or pulmonary embolism, prophylaxis of deep vein thrombosis in patients who have undergone hip or knee replacement surgery, and to reduce risk of major thrombotic vascular events (such as myocardial infarction or ischemic stroke) in patients with peripheral artery disease.

The most serious side effect of DOAC therapy is uncontrolled bleeding, which is difficult to treat. Currently, a specific reversal agent for apixaban and rivaroxaban is available (andexanet alfa (ANDEXXA®)). However, this reversal agent carries significant risks, including arterial and venous thrombosis, myocardial infarction, ischemic stroke, cardiac arrest, and sudden death.

Factor Xa: Factor X is the vitamin K-dependent coagulation factor X of the blood coagulation cascade. This factor undergoes multiple processing steps before its preproprotein is converted to a mature two-chain form by the excision of the tripeptide RKR. The two chains of the factor are held together by one or more disulfide bonds; the light chain contains two EGF-like domains, while the heavy chain contains the catalytic domain which is structurally homologous to those of the other hemostatic serine proteases. The mature factor (Factor Xa) is activated by the cleavage of the activation peptide by factor IXa (in the intrinsic pathway), or by factor VIIa (in the extrinsic pathway). The activated factor then converts prothrombin to thrombin in the presence of factor Va, Ca²⁺, and phospholipid during blood clotting.

Factor X sequences are publicly known. Exemplary human Factor X nucleic acid and amino acid sequences include GenBank Accession Nos. NM_000504.4 and NP_000495.1, respectively (both of which are incorporated herein by reference as present in the GenBank database as of May 6, 2022). An exemplary Factor X preproprotein amino acid sequence is SEQ ID NO: 1. An exemplary wild type (e.g., unmodified) Factor Xa amino acid sequence is amino acids 41-488 of SEQ ID NO: 1.

Heterologous: A heterologous protein, polypeptide or nucleic acid refers to a protein, polypeptide or nucleic acid derived from a different source or species. A heterologous protein or polypeptide may also refer to a protein or polypeptide with an amino acid sequence that differs from a naturally occurring protein or polypeptide. Similarly, a heterologous nucleic acid refers to a nucleic acid with a nucleotide sequence that differs from a naturally occurring nucleic acid molecule.

Isolated: An “isolated” biological component (such as a nucleic acid molecule, protein, or cell) has been substantially separated or purified away from other biological components in the cell or tissue of an organism, or the organism itself, in which the component occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins. Isolated does not require absolute purity, and can include protein, peptide, or nucleic acid molecules that are at least 50% isolated, such as at least 75%, 80%, 90%, 95%, 98%, 99%, or even 99.9% isolated.

Pharmaceutically acceptable carrier: Remington: The Science and Practice of Pharmacy, Adejare (Ed.), Academic Press, London, United Kingdom, 23^{rd} Edition (2021), describes compositions and formulations suitable for pharmaceutical delivery of one or more therapeutic compounds, molecules or agents (e.g. polypeptides).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide or protein: A polymer in which the monomers are amino acid residues which are joined together through amide bonds. When the amino acids are alpha-amino acids, either the L-optical isomer or the D-optical isomer can be used. The terms “polypeptide,” “peptide” and “protein” are used interchangeably herein. These terms apply to amino acid polymers in which one or more amino acid residue is an artificial chemical mimetic of a corresponding naturally occurring amino acid, as well as to naturally occurring amino acid polymers and non-naturally occurring amino acid polymers. The term “residue” or “amino acid residue” includes reference to an amino acid that is incorporated into a protein, polypeptide, or peptide.

A conservative substitution in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4, 5, 6, 7, 8, 9, or 10 substitutions) retains the structure and function of the corresponding protein or peptide without the conservative substitution. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing protein activity or binding affinity (such as thrombin cleavage activity or apixaban binding affinity).

Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Recombinant: A recombinant nucleic acid molecule or protein is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques. The term “recombinant” also includes nucleic acids and proteins that have been altered solely by addition, substitution, or deletion of a portion of the natural nucleic acid molecule or protein.

Subject: Living multi-cellular vertebrate organisms, a category that includes human and non-human mammals. In some aspects herein, the subject is a human, veterinary, or laboratory subject.

Therapeutically effective amount or effective amount: The amount of an agent, such as a nucleic acid, polypeptide, or other therapeutic agent, that is sufficient to prevent, treat, reduce, and/or ameliorate the symptoms and/or underlying causes of a disorder or disease. In some aspects, an “effective amount” is an amount that is sufficient to decrease or reverse Factor Xa inhibition (for example by a DOAC) and/or to treat or inhibit bleeding due to DOAC therapy in a subject.

Treating or ameliorating a condition: “Treating” refers to a therapeutic intervention that ameliorates a sign or symptom of a disease or pathological condition after it has begun to develop. “Ameliorating” refers to the reduction in the number or severity of signs or symptoms of a disease or pathological condition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes.

### II. Modified Factor Xa Polypeptides and Nucleic Acids

The present disclosure describes the design of modified Factor Xa polypeptides. In some aspects, the modified Factor Xa polypeptides have at least about 300% (for example, at least about 300%, at least about 325%, at least about 350%, at least about 375%, at least about 400%, at least about 425%, at least about 450%, or more, such as about 300-450%, about 325-375%, about 350-400%, about 375-425%, or about 400-450%) of the enzymatic activity of unmodified Factor Xa in the presence of apixaban. In some examples, the enzymatic activity of Factor Xa is measured using thrombin generation assay. An exemplary thrombin generation assay is described in Example 1; however, other thrombin generation assays are available and can be utilized by one of ordinary skill in the art.

In other aspects, the modified Factor Xa polypeptides have at least about 50-fold higher (for example, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 250-fold, at least about 300-fold, at least about 350-fold, at least about 400-fold higher, or more, such as about 50-100-fold higher, about 75-150-fold higher, about 125-175-fold higher, about 150-200-fold higher, about 200-250-fold higher, about 225-275-fold higher, about 250-300-fold higher, about 300-350-fold higher, or about 350-400-fold higher) IC₅₀ values for apixaban compared to unmodified Factor Xa. These polypeptides can be utilized to reverse or decrease the effects of DOACs (such as apixaban) when required, such as in the case of bleeding (such as uncontrolled or major bleeding) in a subject who has been treated with DOAC therapy.

In some aspects, the modified Factor Xa polypeptides disclosed herein include substitution of at least one amino acid (such as at least one, at least two, at least three, at least four, at least five, at least six, at least seven, at least eight, at least nine, at least ten, or more amino acids) compared to a wild type or unmodified Factor Xa polypeptide. In some examples, the unmodified Factor Xa polypeptide is amino acids 41-488 of SEQ ID NO: 1. In particular examples, the Factor Xa polypeptide is a human Factor Xa polypeptide.

Factor Xa is composed of four main subsites, designated S1, S2, S3, and S4 (see, e.g., Zacconi, in Anticoagulant Drugs, Ed. Bozic-Mijovski, Intech Open, 2018, pp. 11-37; Hsu et al., J. Biol. Chem. 283:12343-12353, 2008). In some aspects, a disclosed modified Factor Xa polypeptide includes substitution of one or more amino acids in the S4 subsite (S4 binding pocket) of Factor Xa. In other aspects, a disclosed modified Factor Xa polypeptide includes substitution of one or more amino acids in the S1 subsite (S1 binding pocket) of Factor Xa. In further aspects, a disclosed modified Factor Xa polypeptide includes substitution of one or more amino acids in each of the S1 subsite and the S4 subsite.

In some aspects, the modified Factor Xa polypeptides include polypeptides with one or more of the amino acid substitutions listed in Table 1. In some examples, the modified Factor Xa polypeptide includes one or more amino acid substitutions selected from W439A, 312-317NRFTKE→STYVPG, G450A, G440A, C415A+C443A, 312-317NRFTKE→KNYQRD, and W439A+312-317NRFTKE→STYVPG (amino acid numbering corresponding to Factor X amino acid sequence of SEQ ID NO: 1). In some aspects, the modified Factor Xa polypeptide includes one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and has an amino acid sequence at least 95%, at least 96%, at least 97%, at least 98%, or at least 99% identical to the amino acid sequence of any one of SEQ ID NOs: 2, 4, 6, 8, 10, 12, and 14. In some examples, the modified Factor Xa polypeptide has an amino acid sequence including or consisting of any one of SEQ ID NOs: 2, 4, 6, 8, 10, 12, and 14. In other aspects, the modified Factor Xa polypeptide includes one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and has an amino acid sequence at least 95%, at least 96%, at least 97%, at least 98%, or at least 99% identical to amino acids 3-450 of any one of SEQ ID NOs: 2, 4, 6, 8, 10, 12, and 14, or includes or consists of the amino acid sequence of amino acids 3-450 of any one of SEQ ID NOs: 2, 4, 6, 8, 10, 12, and 14.

TABLE 1
Exemplary modified Factor Xa polypeptides

| | | Factor Xa | Amino acid | Nucleic acid |
|---|---|---|---|---|
| Name | Amino acid substitution(s)* | subsite | SEQ ID NO: | SEQ ID NO: |
| Variant 61 | W439A | S4 | 2 | 3 |
| Variant 70 | Amino acids 312-317 | S4 | 4 | 5 |
| | replaced with STYVPG | | | |
| Variant 63 | G450A | S1 | 6 | 7 |
| Variant 65 | G440A | S1 | 8 | 9 |
| Variant 68 | C415A + C443A | S1 | 10 | 11 |
| Variant 69 | Amino acids 312-317 | S4 | 12 | 13 |
| | replaced with KNYQPD | | | |
| Variant 73 | W439A + amino acids 312- | S4 | 14 | 15 |
| | 317 replaced with STYVPG | | | |

*Numbering corresponds to position in Factor X preproprotein (e.g., SEQ ID NO: 1)

Additional minor alterations of the modified Factor Xa polypeptides provided herein are also contemplated. Such alterations may result in polypeptides that have substantially equivalent activity and/or binding affinity as compared to the counterpart starting polypeptide. Such alterations may be deliberate, for example as by site-directed mutagenesis, or may be spontaneous. All of the polypeptides produced by these alterations are included herein. Thus, a specific, non-limiting example is a conservative variant of a modified Factor Xa polypeptide (such as a conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). In other examples, the protein may include one or more non-conservative substitutions (for example 1-10 non-conservative substitutions, 2-5 non-conservative substitutions, 4-9 non-conservative substitutions, such as 1, 2, 5 or 10 non-conservative substitutions), so long as the polypeptide retains similar thrombin cleavage activity and/or apixaban affinity to the starting polypeptide.

The disclosed polypeptides can be prepared by chemical synthesis or isolated by methods including preparative chromatography and immunological separations. Polypeptides can also be produced using molecular genetic techniques, such as by inserting a nucleic acid encoding the polypeptide into an expression vector, introducing the expression vector into a host cell (such as E. coli or mammalian cells), and isolating the polypeptide. In some examples, the protein includes a tag (such as an N-terminal or C-terminal tag), for example for use in protein purification. Exemplary tags include a His-tag, a GST tag, an antibody recognition sequence (such as a Myc-tag or HA-tag), or protein A. In some aspects, the polypeptide is produced by bacteria (such as E. coli) or mammalian cells expressing the polypeptide from an expression vector (such as a vector including a constitutive or a regulatable promoter).

In some examples, the modified Factor Xa polypeptide does not include the signal peptide and propeptide sequence of Factor X (e.g., amino acids 1-40 of SEQ ID NO: 1). Thus, in some examples, the disclosed modified Factor Xa polypeptides do not include a starting methionine (e.g., beginning at amino acid 41 of SEQ ID NO: 1). In other examples, the modified Factor Xa polypeptide is expressed with a N-terminal tag, which is subsequently cleaved prior to use, and one or more amino acids may remain at the N-terminal of the polypeptide as a result of the cleavage site. In one example, the modified Factor Xa polypeptide is expressed with an N-terminal protein A tag, which is removed by proteolytic cleavage. In such examples, the polypeptide includes an N-terminal glycine-proline (GP), as a result of the protein cleavage site.

Further provided are nucleic acid molecules (e.g., DNA, cDNA, RNA or mRNA) encoding the modified Factor Xa polypeptides disclosed herein. Unless otherwise specified, a “nucleic acid molecule encoding a polypeptide” includes all nucleotide sequences that are degenerate versions of each other and encode the same amino acid sequence. For example, a polynucleotide encoding a disclosed modified Factor Xa polypeptide includes a nucleic acid sequence that is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged. In some aspects, the disclosed polypeptide sequences are back-translated to codon optimized DNA using standard methods.

In some aspects, the nucleic acid encodes a polypeptide including one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and having an amino acid sequence at least 95%, at least 96%, at least 97%, at least 98%, or at least 99% identical to the amino acid sequence of any one of SEQ ID NOs: 2, 4, 6, 8, 10, 12, and 14. In some examples, the nucleic acid encodes a polypeptide including or consisting of any one of SEQ ID NOs: 2, 4, 6, 8, 10, 12, and 14. In some examples, the nucleic acid encodes a polypeptide including one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and has a nucleotide sequence at least 95%, at least 96%, at least 97%, at least 98%, or at least 99% identical to any one of SEQ ID NOs: 3, 5, 7, 9, 11, 13, and 15. In other examples the nucleic acid has a nucleotide sequence including or consisting of any one of SEQ ID NOs: 3, 5, 7, 9, 11, 13, and 15.

In other aspects, the nucleic acid encodes a polypeptide including one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and having an amino acid sequence at least 95%, at least 96%, at least 97%, at least 98%, or at least 99% identical to amino acids 3-450 of any one of SEQ ID NOs: 2, 4, 6, 8, 10, 12, and 14. In some examples, the nucleic acid encodes a polypeptide including or consisting of amino acids 3-450 any one of SEQ ID NOs: 2, 4, 6, 8, 10, 12, and 14. In some examples, the nucleic acid encodes a polypeptide including one or more amino acid substitutions selected from W439A, 312-317NRFTKE→STYVPG, G450A, G440A, C415A+C443A, 312-317NRFTKE→KNYQRD, and W439A+312-317NRFTKE→STYVPG and has a nucleotide sequence at least 95%, at least 96%, at least 97%, at least 98%, or at least 99% identical to nucleotides 7-1350 of any one of SEQ ID NOs: 3, 5, 7, 9, 11, 13, and 15. In other examples the nucleic acid has a nucleotide sequence including or consisting of nucleotides 7-1350 of any one of SEQ ID NOs: 3, 5, 7, 9, 11, 13, and 15.

Minor alterations of nucleic acids encoding a modified Factor Xa polypeptide primary amino acid sequence are also contemplated herein. Such alterations to the nucleic acid may result in polypeptides that have substantially equivalent activity as compared to the starting counterpart polypeptide described herein. Such alterations may be deliberate, for example as by site-directed mutagenesis, or may be spontaneous. All of the nucleic acids produced by these alterations are included herein. Thus, a specific, non-limiting example of an altered nucleic acid encoding a disclosed polypeptide is a nucleic acid encoding a conservative variant of the polypeptide (such as encoding a conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). In other examples, the nucleic acid may encode a polypeptide including one or more non-conservative substitutions (for example, encoding 1-10 non-conservative substitutions, 2-5 non-conservative substitutions, 4-9 non-conservative substitutions, such as 1, 2, 5 or 10 non-conservative substitutions), so long as the encoded polypeptide retains similar thrombin cleavage activity and/or apixaban affinity to the starting polypeptide.

Vectors that include the disclosed nucleic acid molecules are also provided. DNA sequences encoding the disclosed polypeptides can be expressed in vitro or in vivo by DNA transfer into a suitable host cell. The cell may be prokaryotic or eukaryotic. Methods of stable transfer, meaning that the foreign DNA is continuously maintained in the host, are known in the art. Polynucleotide sequences encoding the disclosed polypeptides can be operably linked to expression control sequences, such as heterologous expression control sequences (such as a heterologous promoter). An expression control sequence operably linked to a coding sequence is joined such that expression of the coding sequence is achieved under conditions compatible with the expression control sequences. The expression control sequences include, but are not limited to, one or more appropriate promoters, enhancers, transcription terminators, a start codon in front of a protein-encoding gene, splicing signal for introns, maintenance of the correct reading frame of that gene to permit proper translation of mRNA, and stop codons.

Hosts can include microbial, yeast, insect, and mammalian organisms. Methods of expressing DNA sequences having eukaryotic or viral sequences in prokaryotes are well known in the art. Non-limiting examples of suitable host cells include bacteria, archaea, insect, fungi (for example, yeast), plant, and animal cells (for example, mammalian cells, such as human cells). Exemplary cells of use include Escherichia coli, Bacillus subtilis, Saccharomyces cerevisiae, Salmonella typhimurium, SF9 cells, C129 cells, Neurospora, and immortalized mammalian myeloid and lymphoid cell lines. Techniques for the propagation of mammalian cells in culture are known to one of ordinary skill in the art. Examples of commonly used mammalian host cell lines are VERO cells, HeLa cells, CHO cells, HEK 293 cells, WI38 cells, BHK cells (such as BHK21 cells), HT-1080 cells, PER.C6 cells, HKB-11 cells, HuH-7 cells, and COS cells, although other cell lines may be used, such as cells designed to provide higher expression, desirable glycosylation patterns, or other features.

Transformation of a host cell with recombinant DNA can be carried out by techniques known to those skilled in the art. Where the host is prokaryotic, such as, but not limited to, E. coli, competent cells which are capable of DNA uptake can be prepared from cells harvested after exponential growth phase and subsequently treated by the CaCl₂) method using procedures known in the art. Alternatively, MgCl₂ or RbCl can be used. Transformation can also be performed after forming a protoplast of the host cell if desired, or by electroporation. When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with a polynucleotide encoding disclosed polypeptide and a second foreign DNA molecule encoding a selectable phenotype, such as the herpes simplex thymidine kinase gene. Another method is to use a eukaryotic viral vector, such as simian virus 40 (SV40) or bovine papilloma virus, to infect or transform eukaryotic cells and express the protein.

### III. Compositions and Methods of Treatment

Disclosed herein are methods of treating or inhibiting bleeding in a subject who is receiving or has received direct oral anticoagulant therapy, using the modified Factor Xa polypeptides described herein. In some aspects, the methods include administering to the subject an effective amount of a disclosed modified Factor Xa polypeptide or a composition including an effective amount of a disclosed modified Factor Xa polypeptide. In some examples, the subject receiving DOAC therapy is a subject receiving a Factor Xa inhibitor DOAC, such as apixaban, rivaroxaban, edoxaban, betrixaban, darexaban, otamixaban, letaxaban, LY517717, or GW813893. In one specific example, the subject is receiving treatment with apixaban.

In some aspects, treatment with a disclosed modified Factor Xa polypeptide or composition reduces or inhibits bleeding in a subject receiving DOAC therapy, for example an uncontrolled or major bleeding event. In some examples, the subject has received at least one dose of DOAC within the previous 24 hours. In some examples, the subject is undergoing or will undergo a surgical procedure (such as an emergency procedure). In other examples, the subject has experienced an injury or trauma (including, but not limited to a traumatic brain injury, or an injury that requires emergency surgery).

The treatment may reduce bleeding time by at least about 10%, at least about 20%, at least about 30%, at least about 40%, at least about 50%, at least about 60%, at least about 70%, at least about 80%, at least about 90%, or more, compared to a control (such as a subject receiving DOAC therapy without treatment with a disclosed modified Factor Xa polypeptide or composition). The treatment may reduce blood loss by at least about 10%, at least about 20%, at least about 30%, at least about 40%, at least about 50%, at least about 60%, at least about 70%, at least about 80%, at least about 90%, or more, compared to a control (such as a subject receiving DOAC therapy without treatment with a disclosed modified Factor Xa polypeptide or composition). In some examples, the treatment reduces bleeding time by about 60% and/or reduces blood loss by about 90% compared to a control (such as a subject receiving DOAC therapy without treatment with a disclosed modified Factor Xa polypeptide).

In other examples, the treatment provides effective clinical hemostasis in the subject, such as non-visible bleeding effective hemostasis (e.g., stable hemoglobin level at 48 hours after initial treatment), visible bleeding effective hemostasis (e.g., no visible bleeding with 4 hours of treatment), musculoskeletal effective bleeding hemostasis (e.g., reduced pain and swelling within 24 hours), or intracranial effective hemostasis (e.g., stable hematoma or increased by <35% compared with baseline within 12 hours). In other examples, effective clinical hemostasis includes no need for further infusion of hemostatic agents, coagulation factors or transfusion of blood products by 48 hours after initial treatment. See, e.g., Khorsand et al., Journal of Thrombosis and Haemostasis 14:211-214, 2015.

Pharmaceutical compositions including the disclosed polypeptides are also provided. The pharmaceutical compositions can include one or more modified Factor Xa polypeptides and one or more pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure. Remington: The Science and Practice of Pharmacy, Adejare (Ed.), Academic Press, London, United Kingdom, 23^{rd} Edition (2021), describes compositions and formulations suitable for pharmaceutical delivery of one or more therapeutic agents

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Compositions for oral administration include powders or granules, suspensions or solutions in water or non-aqueous media, capsules, sachets, or tablets. Thickeners, flavorings, diluents, emulsifiers, dispersing aids or binders may be desirable.

In some aspects, an effective amount of a modified polypeptide or composition disclosed herein is administered to a subject. In some examples, an effective amount of the modified Factor Xa polypeptide or composition is administered, such as about 0.05 mg/kg to about 10 mg/kg of the modified polypeptide. In some examples, the subject is administered about 0.05 mg/kg to about 0.25 mg/kg, about 0.1 mg/kg to about 0.5 mg/kg, about 0.25 mg/kg to about 0.75 mg/kg, about 0.5 mg/kg to about 1 mg/kg, about 0.75 mg/kg to about 1.5 mg/kg, about 1 mg/kg to about 2.5 mg/kg, about 2.5 mg/kg to about 7.5 mg/kg, or about 5 mg/kg to about 10 mg/kg (such as about 0.05 mg/kg, about 0.075 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In one example, about 1 mg/kg of the modified polypeptide is administered to the subject.

In other examples, the polypeptide or composition can be provided in unit dosage form for administration to a subject. A unit dosage form contains a suitable single preselected dosage for administration to the subject, suitable marked or measured multiples of two or more preselected unit dosages, and/or a metering mechanism for administering the unit dose or multiples thereof. In some examples, a the polypeptide or composition is provided in a unit dose of about 2.5 mg to about 1.5 g (such as about 2.5 mg to about 7.5 mg, about 5 mg to about 15 mg, about 10 mg to about 25 mg, about 20 mg to about 80 mg, about 75 mg to about 100 mg, about 100 mg to about 250 mg, about 200 mg to about 500 mg, about 250 mg to about 800 mg, about 500 mg to about 1 g, about 800 mg to about 1.25 g, or about 1 g to about 1.5 g). In some examples, the unit dose is about 5 mg, about 10 mg, about 20 mg, about 25 mg, about 40 mg, about 50 mg, about 75 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 600 mg, about 700 mg, about 800 mg, about 900 mg, about 1 g, about 1.25 g, or about 1.5 g.

The disclosed modified polypeptides or compositions are typically administered parenterally (e.g., intravenously). One of skill in the art can determine appropriate routes of administration. Multiple doses (such as 1, 2, 3, or more doses) of the polypeptides or compositions can be administered, for example, to control continued bleeding. A skilled clinician can select an appropriate administration route and schedule based on clinical studies, the particular subject, clinical situation, and other factors.

### EXAMPLES

The following examples are provided to illustrate particular features of certain aspects of the disclosure, but the scope of the claims should not be limited to those features exemplified.

### Example 1

### Materials and Methods

RosettaFastRelax: Preparation of input structure: The crystal structure of the Factor Xa (PDB ID: 2P16, after apixaban removal) was used as the starting structure for an all-atom refinement process using the Rosetta Relax protocol. The structure was run through fixed backbone Rosetta energy minimization (FastRelax protocol, 1000 decoys generated) and the top-scoring total energy decoy was used for further design. Another FastRelax protocol was performed with the inhibitor apixaban included in the complex and similarly the top-scoring total energy decoy was used for further delta energy analysis.

RosettaDesign: Design of empty apixaban binding pocket: Two rounds of sequence design were used. The purpose of the first was to identify mutations that were deleterious to activity or overexpression in human cells. The goal of the second was to minimize protein destabilization due to aggressive mutagenesis while maintaining the Factor Xa activity and preventing apixaban binding. The first round of designs used the RosettaDesign protocol where all the residues 8 Å around apixaban including the residues in the binding pocket were designated as the “cavity” (excluding the catalytic triad necessary for Factor Xa activity). The 19 top-scoring delta “cavity” designs were identified, and their sequence profiles analyzed. The sequence profiles were threaded with the FastRelax protocol (using resfiles) onto the Factor Xa apixaban complex structure to construct final models. The final models contained the mutations identified previously but in the presence of apixaban to see if they could still fit in a filled binding pocket and to identify clashes with apixaban. Through manual inspection of the models, with or without apixaban, mutations were either reverted or kept, resulting in 21 designs. These 21 designs were experimentally validated and tested. Based on the results from those designs, the second round of design was performed.

The second round entailed using the experimental results and suggested binding pocket locations from Rosetta to create conservative single- and double-point mutants. This conservative approach focused on introducing small changes such as focusing on smaller side-chained amino acids such as Alanine and Valine, relative to their corresponding wild-type amino acid or introducing a much bulkier side chain such as Tryptophan. This conservative approach allowed for identifying single point mutations that could potentially make a difference in apixaban binding and resulted in 9 total designs for the second round of designs.

Homology based designs: Two proteins were used as homology models to identify potential mutations to include as a replacement starting at positions 90 (PDB numbering based on 2P16), 92, 163, and 173. The human Factor VII (PDB ID: 1DAN) crystal structure was superimposed onto the human Factor Xa (PDB ID: 2P16) crystal structure and differences in amino acid identity were analyzed. Final insertion identities were based on proximity to binding pocket, length, and insertion locations were determined based on structure differences. Human Factor VII was chosen based on overall structure similarity and previously unpublished NMR results indicating that it did not bind to apixaban. This resulted in 7 designs during the first round of designs using only the human Factor VII. After experimental expression issues, a conservative second round of homology designs was chosen. The insertions from Factor VII were decreased in length and only 1 position insertion was allowed (at position 173). In addition, two more designs were included that originated from the Factor X from Danio rerio (zebra fish). This protein was also chosen as a homology model for identification of potential mutations based on sequence similarity to human Factor Xa. This addition of another protein and decreasing the length of the Factor VII insertion at position 173 resulted in three designs.

FX expression: All variants of recombinant human FX were expressed in human embryonic kidney 293 (HEK293) cells. The expression vector consisted of a CMV promoter, a prolactin signal sequence, and a N-terminal protein-A (PA) tag separated by a PreScission Protease cleavage site. Transfection was performed using Lipofectamine 3000 (Invitrogen) according to the manufacturer's protocol with psPAX2, pMD2.G and transfer plasmid. Undiluted virus was used to transduce HEK293 cells and 48 hours post-transduction FACS (FACS Aria III cell sorter) was used to sort for the population of the GFP positive cells. These cells were expanded and ultimately seeded into an adherent cell bioreactor (Cesco Bioengineering, Taiwan) for long term growth and protein production.

FX Purification: Cell supernatant containing PA-FX was centrifuged for 10 min at 5,000 rpm, filtered through a 0.22 μm membrane, and loaded onto an IgG FF column (Cytiva). The column was extensively washed with 20 mM sodium phosphate pH 7 and then equilibrated with 25 mM HEPES pH 7.5, 250 mM NaCl, and 5% glycerol. PreScission Protease was added into the column at approximately 400 μg/L of supernatant and incubated overnight at 4° C. Cleaved protein was eluted from the column by connecting IgG column to the FPLC system equilibrated in the buffer used for tag cleavage. GST column was connected below IgG to capture PreScission Protease and flow-thru containing FX was collected. Purified recombinant FX was activated with RVV-X (Haematologic Technologies, Inc.), isolated by size-exclusion chromatography on a Superdex 75 Increase 10/300 GL column (Cytiva), and stored at −20° C. in PBS containing 50% (v/v) glycerol.

Chromogenic FXa activity assay: The FXa activity assay based on CS11(65)a chromogenic substrate (Biophen) was performed as described below. FXa samples with or without apixaban were diluted in Tris-BSA buffer containing 5 mM CaCl2 (pH 7.4) and mixed with substrate (300 μM final concentration [f.c.]) at a ratio of 3:2, and absorbance (410 nm) was measured kinetically on a Biotek Synergy H4 microplate reader at 37° C. For each sample, substrate cleavage was calculated by taking the average rate over the time frame between 1 and 2 minutes.

Thrombin generation assay: The procoagulant activity of FXa variants was evaluated by a previously described thrombin generation assay in tissue factor (TF)-triggered pooled normal human plasma (Affinity Biologicals Inc, Ontario, Canada). FXa variant samples were serially diluted in Tris-BSA buffer (pH 7.4) and mixed with a recalcified plasma mixture containing plasma (50% vol/vol f.c.), apixaban, platelet substitute in the form of phosphatidyl choline, phosphatidyl serine, and sphingomyelin phospholipid vesicles (4 μM f.c.; Phospholipid-TGT®; Rossix, Mölndal, Sweden), recombinant lipidated TF (5 pM f.c.; RecombiPlasTin®, Instrumentation Laboratory Company, Lexington, MA, USA), fluorogenic substrate for thrombin ZGGR-AMC (800 μM f.c.; Bachem, Torrance, CA, USA) and calcium chloride (12.5 mM) with a robotic 96-channel pipettor (ViaFlo 96; Integra Biosciences, Bedford, MA, USA). Fluorescence (product of thrombin substrate consumption) was recorded in a kinetic mode at 37° C. using a Tecan Infinite F500 every 45 seconds for a period of 1.7 hours. In-house software was used to calculate calibrate thrombin generation assay (using an internal thrombin calibrator CAT® from Stago, Parsippany, NJ, USA) and calculate thrombin peak height parameter.

Tail clip bleeding model: Animal experiments were performed at the U.S. FDA/CBER Division of Veterinary Services in accordance with the protocol approved by the institutional animal care and use committee (IACUC). Male CD-1 mice (Charles River Laboratories, Kingston, NY, USA) were 5-8 weeks old weighing 28-34 grams. Mice were anesthetized with a mixture of ketamine/xylazine and administered with apixaban or sterile DMSO in saline (NaCl, control) followed 5 minutes later by a FXa preparation via two retro-orbital injections. After 5 minutes, 3 mm of tail were cut, after which the tail was immersed in a tube with predefined volume of 0.9% NaCl at 37° C. and allowed to bleed for 30 minutes under close observation. Blood loss was determined by measuring hemoglobin content and expressed as μl/mouse.

### Example 2

### In Vitro Testing of FXa Variants

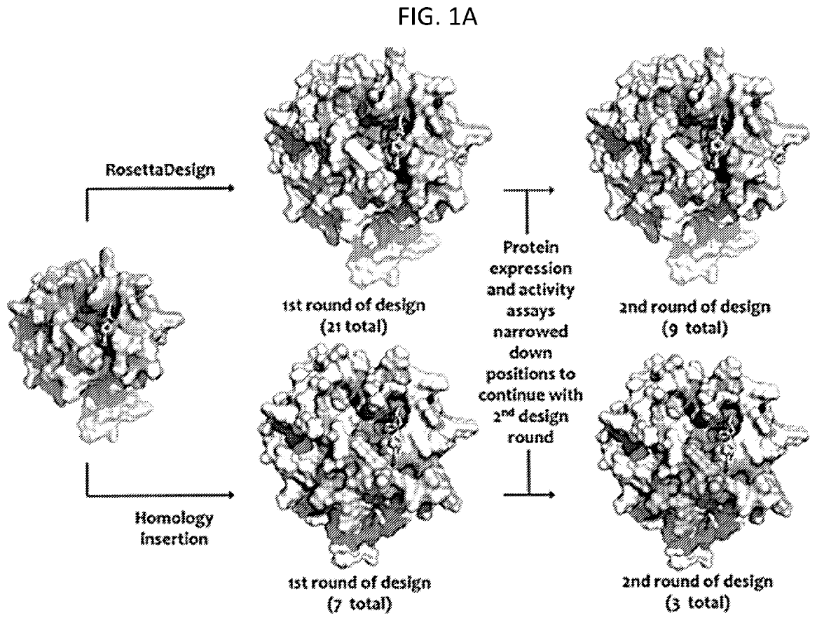

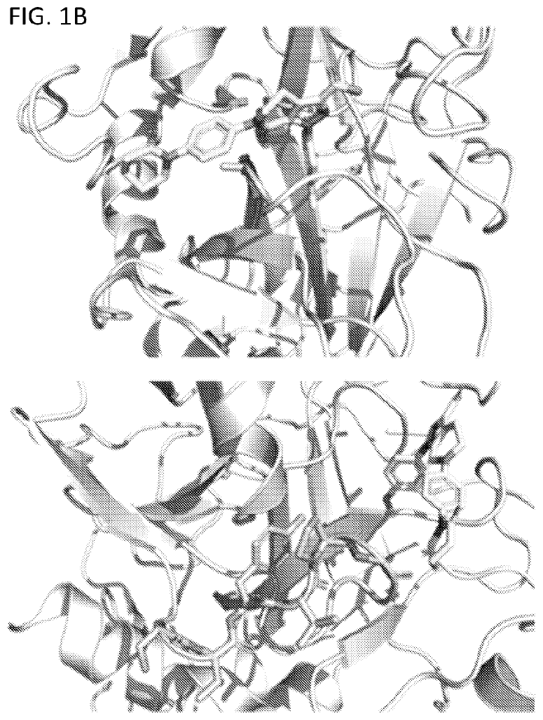

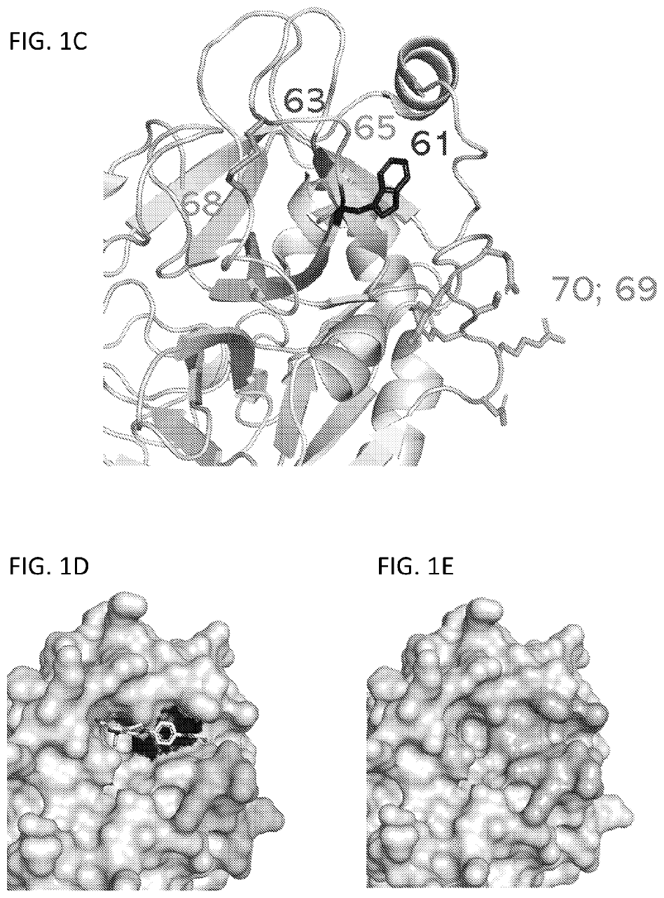

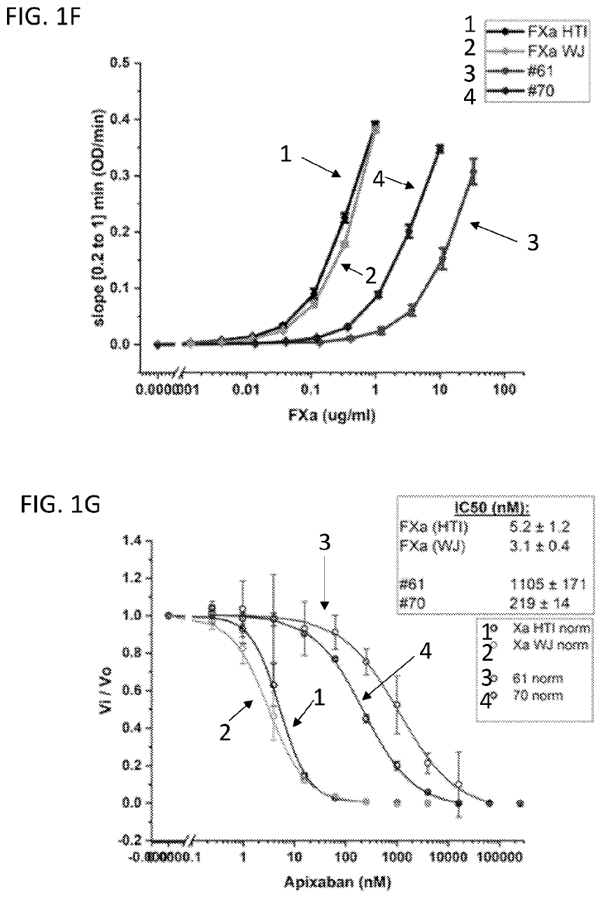

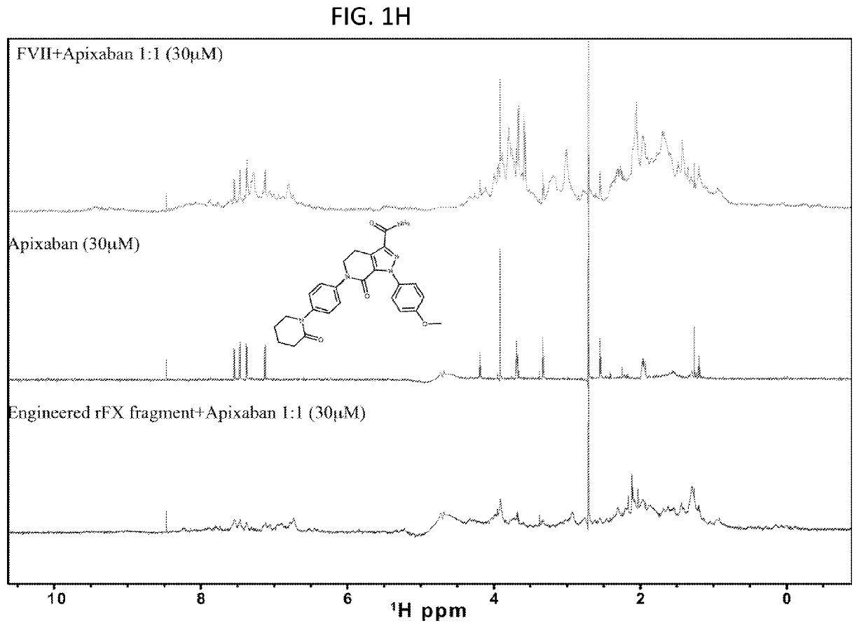

The design of functional FXa analogs for which DOACs have low affinity offer an alternative reversal strategy. In this study, a novel computational approach (FIG. 1A) was used to design functional FXa analogs to which apixaban (the most widely prescribed FXa inhibitor) would have low affinity.

The first round of designs used two strategies: (i) The RosettaDesign protocol where all the residues 8 Å around apixaban including the residues in the binding pocket (except the catalytic triad required for Factor Xa activity) were designated as the “cavity.” The 19 top-scoring delta “cavity” designs were threaded with the FastRelax protocol (using resfiles) onto the FXa-apixaban complex structure to construct final models. The final models allowed identification of clashes with apixaban. Following manual inspection of the models, 21 designs were selected. The second round of designs entailed the use of experimental results from the first round of designs and focused on identification of a single point mutations that could potentially offer the best balance between activity and resistance to apixaban. The second round resulted in of 9 low affinity FXa variants. (ii) Two proteins were used as homology models to identify potential mutations to include as insertions. The human factor VII (FVII) (PDB ID: 1DAN) crystal structure was superimposed onto the human FXa (PDB ID: 2P16) crystal structure and differences in amino acid identity were analyzed. Final insertion identities were based on proximity to binding pocket and length. FVII was chosen for mutational substitutions based on overall structure similarity and NMR results indicating that apixaban does not bind to FVII (FIG. 1H). Seven FXa variants were selected based on this strategy. Some of these designs failed experimental validations. In a follow up round of homology-based designs, the insertions from Factor VII were decreased in length and number. Additionally, amino acid substitutions that originated from the FX of Danio rerio (zebrafish) were included. Using sequences from two proteins homologous to human FXa that do not provide binding pockets for apixaban and calibrating the number of substitutions resulted in three homology insert designs.

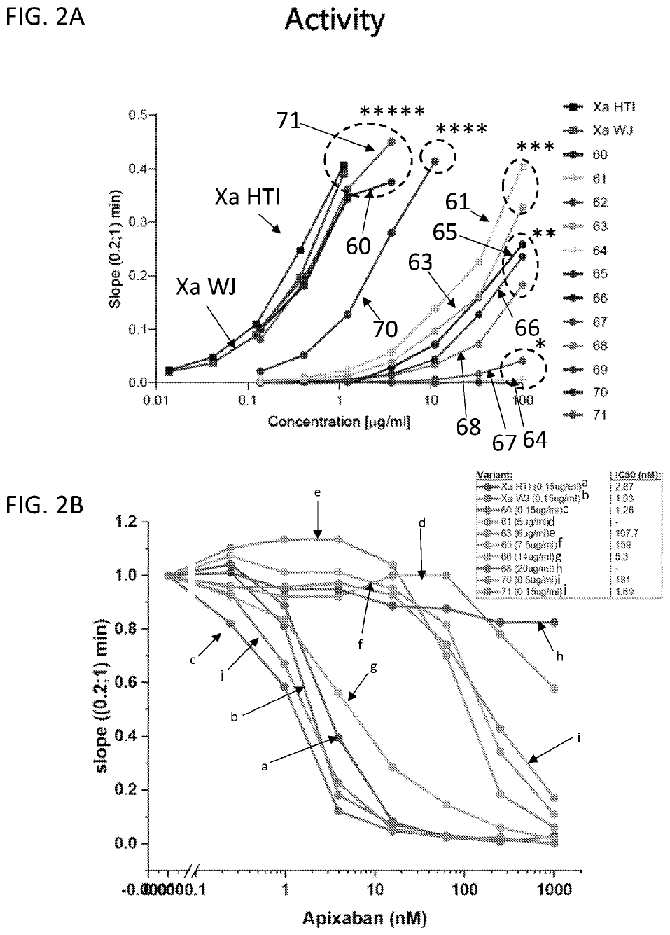

The nine variants were expressed by HEK 293T cells, purified, and demonstrated to be functional in a FXa-substrate cleavage assay. Inhibition of FXa activity by apixaban (IC₅₀) for each of the variants is shown in FIG. 2B. The variants (variant 61 and variant 70) with the highest IC₅₀ values (those that showed the least inhibition by apixaban) were selected for additional characterization.

The two variants selected for additional characterization included the single point mutation variant W215A (variant 61) and insertion variant H91-STYVPG-T98 (variant 70). Additionally, Rosetta Models of variant 61 and variant 70 in complex with apixaban showed that modified regions of the FXa binding pocket are destabilized that cause disruption of proper docking of apixaban into the active site and partial loss of binding.

Using a thrombin generation assay it was shown that although variant 61 and variant 70 were less potent than wild type FXa, both variants were functional (FIG. 1F). Importantly however, both variant 61 and variant 70 exhibited ˜350- and ˜70-fold higher IC₅₀ values for apixaban compared to the wild-type FXa (FIG. 1G). Taken together these data suggest that at clinically relevant concentrations of apixaban, these variant FXa molecules continue to show procoagulant activity.

Activity and inhibitory concentration were determined for a larger series of variants (FIGS. 2A and 2B). Activities for all variants were determined using the rate of chromogenic substrate cleavage in pure buffer system at different FXa concentrations. Variants were assigned into five brackets (FIG. 2A). IC₅₀ for all variants were determined using the rate of chromogenic substrate cleavage by fixed amounts of FXa variants in the presence of increasing apixaban concentrations. Variants were assigned into five brackets based on the IC₅₀ values; <5 nM (*), 5-50 nM (**), 51-150 nM (***), 151-250 (****), >251 nM (*****). Taking a balance between activity and IC₅₀ values (Table 2), six potentially beneficial variants were identified. To be assigned as potentially beneficial the combined scores were ***** but the individual score could not be less than **.

TABLE 2
Activity and IC50 of variants

| | | | | Potential |
|---|---|---|---|---|
| Variant ID | Substitution | Activity | IC50 | benefit? |
| 60 | F396W | ***** | * | No |
| 61 | W439A | *** | ***** | Yes |
| 62 | G450V | * | NA | No |
| 63 | G450A | *** | *** | Yes |
| 64 | G440V | * | NA | No |
| 65 | G440A | ** | **** | Yes |
| 66 | C415A | ** | * | No |
| 67 | C443A | * | NA | No |
| 68 | C415A + C443A | ** | **** | Yes |
| 69 | Amino acids 312-317 | ***** | ** | Yes |
| | replaced with KNYQPD | | | |
| 70 | Amino acids 312-317 | **** | **** | Yes |
| | replaced with STYVPG | | | |
| 71 | Amino acids 395-401 | ***** | * | No |
| | replaced with NFKISGR | | | |

### Example 3

### In Vivo Testing of FXa Variants

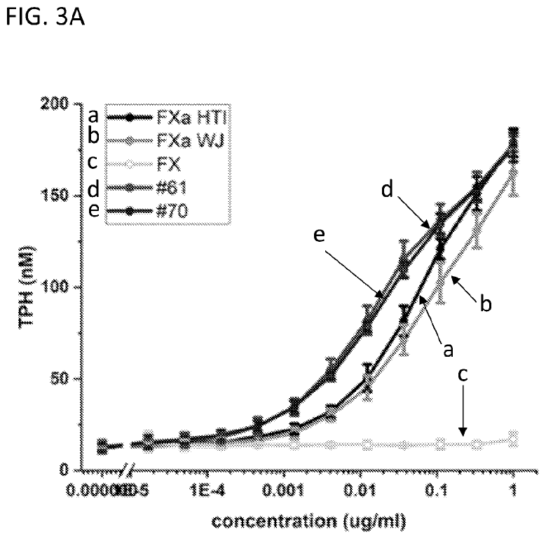

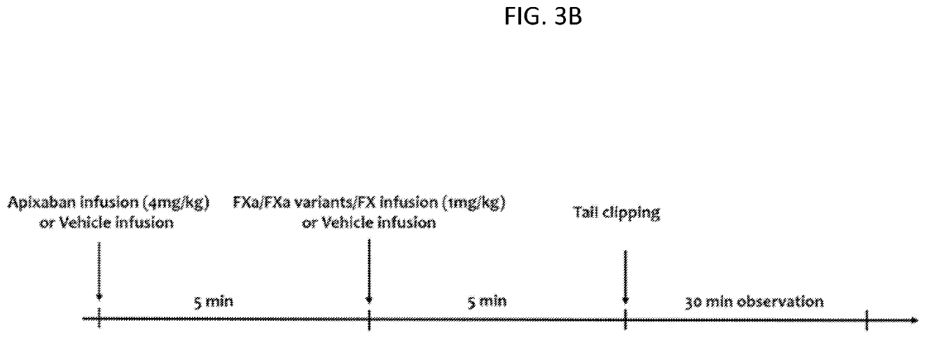

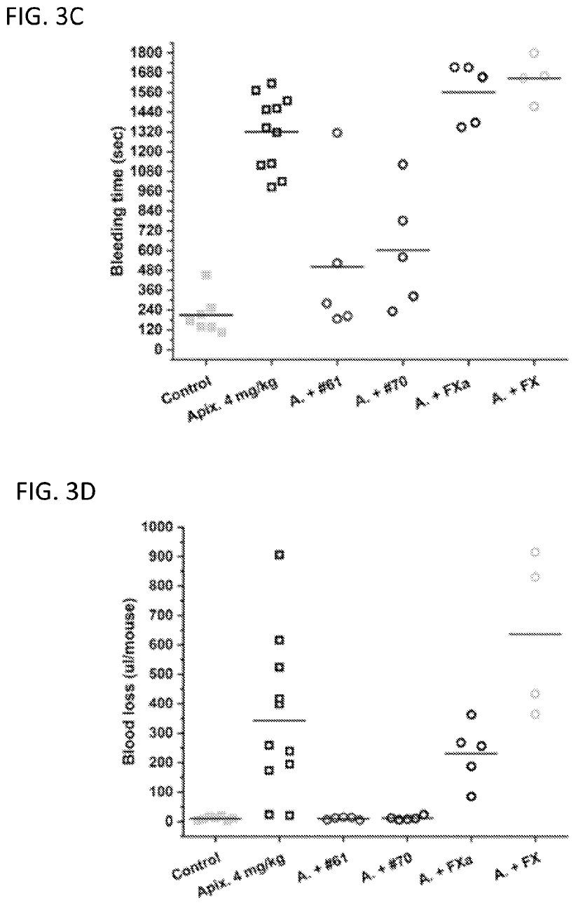

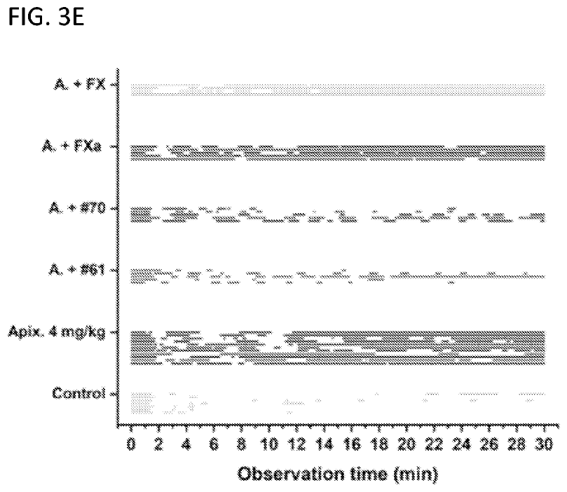

To demonstrate the potential in vivo utility of the engineered FXa variants to bypass the effect of apixaban, the activity of wild type and variant FXa molecules in the presence of a fixed amount (1 μM) of apixaban was measured (FIG. 3A). Although the variant FXa molecules are less potent than wild-type FXa, the apparent Km of wild type FXa is 3.1±0.4 nM compared to 1105±171 nM for variant 61 and 219±14 nM for variant 70.

The design of the in vivo study in mice is depicted in FIG. 3B. Briefly, either control buffer or apixaban was infused into the veins of mice, after 5 min, control buffer, variant 61, variant 70, or wild-type FXa (all at 1 mg/kg) were infused. The tails of the mice were clipped, and bleeding monitored by measuring bleeding time (FIG. 3C) and blood loss (FIG. 3D). Bleeding time was significantly greater in the group treated with apixaban compared to the control group. This observation is consistent with the mechanism of action of apixaban, that is, inhibition of coagulation by binding to FXa. This also shows that the mouse model used by us replicates the clinical challenge associated with the use of apixaban, which is uncontrolled bleeding. Treatment of mice with either FX or FXa following treatment with apixaban did not significantly reduce the bleeding time. However, treatment with either variant 61 or variant 70 significantly reduced bleeding time. Similar results demonstrating that the variant 61 and variant 70 successfully reversed the anticoagulant effect of apixaban were obtained when blood loss was measured rather than bleeding time (FIG. 3D). All bleeding episodes were plotted using the length and number of episodes of bleeds for each individual mouse (FIG. 3E) to determine the bleeding profiles.

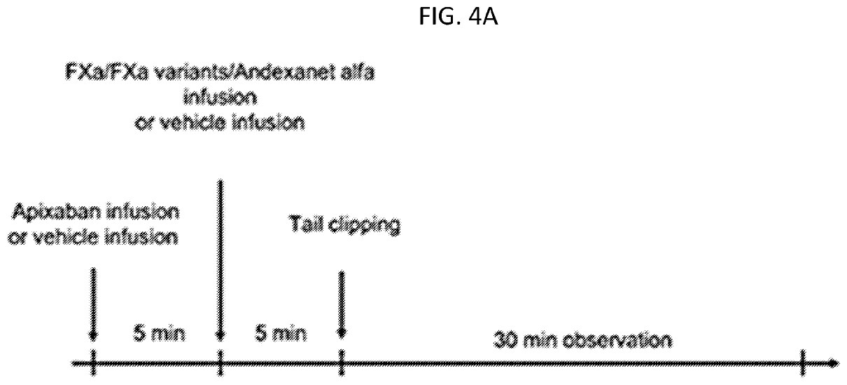

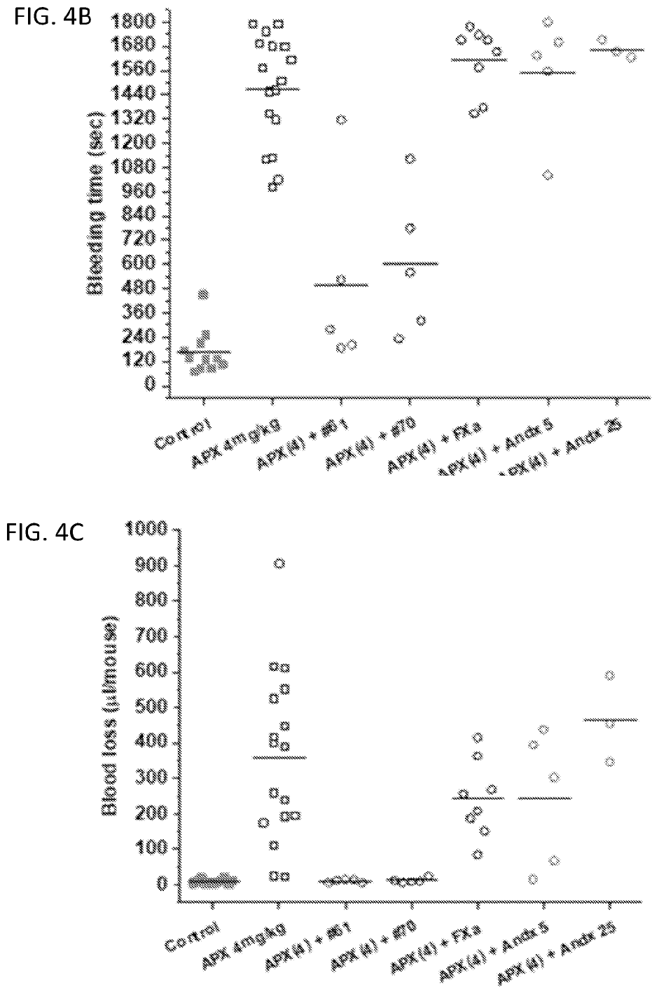

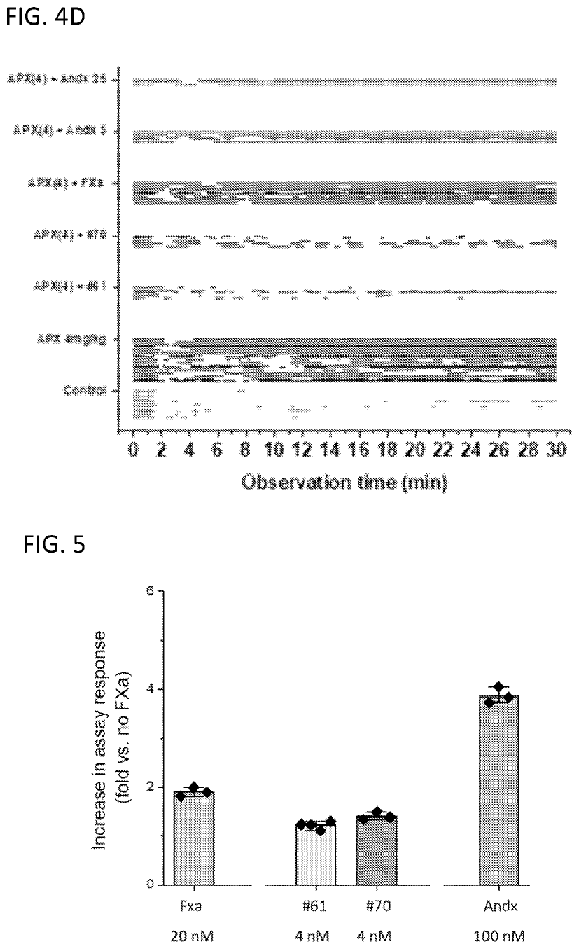

Additional experiments were performed in the mouse model, as above, but with the inclusion of andexanet alfa for comparison. The experimental strategy is depicted in FIG. 4A. Using an apixaban dose of 4 mg/kg that is sufficient to induce bleeding effective reversal of bleeding by variants #61 and #70 was demonstrated. A significant decrease in bleeding time or blood loss was observed in animals treated with #61 and #70 (FIGS. 4B and 4C). Infusion of wild type FXa had no significant effect on bleeding time or blood loss. Andexanet alfa at the doses of 5 mg/kg and 25 mg/kg did not show a hemostatic effect (Andx 25 mg/kg is roughly equivalent to the high-dose clinical regimen of 1760 mg). Bleeding graphs demonstrated that hemostasis mediated by variants #61 and #70 prevented blood loss via multiple breaks in blood flowing from the tail cut (FIG. 4D). Animals treated with apixaban (with or without andexanet alfa or wild type FXa) experienced mostly uninterrupted flow of blood (FIG. 4D).

Tissue factor pathway inhibitor (TFPI) plays a very important role in regulation of the procoagulant activity of tissue factor (TF). Andexanet alfa has been shown to reduce TFPI activity, and since TFPI is a major inhibitor of TF-FVIIa complex, a decrease its activity may result in thrombogenesis. TFPI activity was determined using ACTICHROME® TFPI assay (BioMedica Diagnostics) according to manufacturer's protocol. At physiological concentrations, both variants #61 and #70 showed much smaller response to TPFI compared to andexanet alfa (FIG. 5). Thus, these agents are believed to have minimal risk of thrombogenesis.

It will be apparent that the precise details of the methods or compositions described may be varied or modified without departing from the spirit of the described aspects of the disclosure. We claim all such modifications and variations that fall within the scope and spirit of the claims below.

1. A modified Factor Xa polypeptide comprising:
one or more amino acid substitutions in Factor Xa subsite S1, S4, or both; and
having enzymatic activity of at least 300% of unmodified Factor Xa in the presence of apixaban, IC₅₀ value for apixaban of at least 50-fold higher than unmodified Factor Xa, or both.

2. The modified Factor Xa polypeptide of claim 1, wherein the unmodified Factor Xa comprises the amino acid sequence of SEQ ID NO: 1.

3. The modified Factor Xa polypeptide of claim 1, wherein the modified Factor Xa polypeptide does not include the signal peptide and propeptide of Factor Xa.

4. The modified Factor Xa polypeptide of claim 1, wherein the one or more amino acid substitutions are selected from 312-317NRFTKE→STYVPG, W439A, G450A, G440A, C415A+C443A, 312-317NRFTKE→KNYQRD, and W439A+312-317NRFTKE→STYVPG, wherein the amino acid numbering corresponds to SEQ ID NO: 1.

5. The modified Factor Xa polypeptide of claim 4, wherein the modified polypeptide has an amino acid sequence with at least 95% sequence identity to any one of SEQ ID NOs: 4, 2, 6, 8, 10, 12, and 14.

6. The modified Factor Xa polypeptide of claim 5, wherein the modified polypeptide comprises or consists of the amino acid sequence of any one of SEQ ID NOs: 4, 2, 6, 8, 10, 12, and 14.

7. The modified Factor Xa polypeptide of claim 4, wherein the modified polypeptide has an amino acid sequence with at least 95% sequence identity to amino acids 3-450 of any one of SEQ ID NOs: 4, 2, 6, 8, 10, 12, and 14.

8. The modified Factor Xa polypeptide of claim 7, wherein the modified polypeptide comprises the amino acid sequence of amino acids 3-450 of any one of SEQ ID NOs: 4, 2, 6, 8, 10, 12, and 14.

9. A nucleic acid encoding the modified Factor Xa polypeptide of claim 1.

10. The nucleic acid of claim 9, wherein:
the nucleic acid has a nucleotide sequence with at least 95% sequence identity to any one of SEQ ID NOs: 5, 3, 7, 9, 11, 13, and 15;
the nucleic acid comprises the nucleotide sequence of any one of SEQ ID NOs: 5, 3, 7, 9, 11, 13, and 15; or
the nucleic acid consists of the nucleotide sequence of any one of SEQ ID NOs: 5, 3, 7, 9, 11, 13, and 15.

11. (canceled)

12. The nucleic acid of claim 9, wherein:
the nucleic acid has a nucleotide sequence with at least 95% sequence identity to nucleotides 7-1350 of any one of SEQ ID NOs: 5, 3, 7, 9, 11, 13, and 15;
the nucleic acid comprises the nucleotide sequence of nucleotides 7-1350 of any one of SEQ ID NOs: 5, 3, 7, 9, 11, 13, and 15; or
the nucleic acid consists of the nucleotide sequence of nucleotides 7-1350 of any one of SEQ ID NOs: 5, 3, 7, 9, 11, 13, and 15.

13. (canceled)

14. A vector comprising the nucleic acid of claim 9.

15. A host cell comprising the nucleic acid of claim 9 or a vector comprising the nucleic acid.

16. A composition comprising the modified Factor Xa polypeptide of claim 1, and a pharmaceutically acceptable carrier.

17. A method of treating or inhibiting bleeding in a subject receiving direct oral anticoagulant therapy, the method comprising administering to the subject an effective amount of the modified Factor Xa polypeptide of claim 1, thereby treating or inhibiting the bleeding.

18. The method of claim 17, wherein the direct oral anticoagulant therapy is apixaban, rivaroxaban, edoxaban, or betrixaban.

19. The method of claim 18, wherein the direct oral anticoagulant therapy is apixaban.

20. The method of claim 17, wherein the modified Factor Xa polypeptide or composition is administered intravenously.

21. The method of claim 17, wherein the modified Factor Xa polypeptide or composition is administered at a dose of about 0.1-10 mg/kg.

22. The method of claim 17, wherein the treatment reduces bleeding time by at least 10% compared to a control, reduces blood loss by at least 10% compared to a control, or both.


SEQUENCE LISTING (15 sequences)

SEQ ID NO: 1 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 2 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSAGEGCARKGKYGIYTKVTAFLKWIDRSMKTRGLPKAKSHAPEVITSSPLK

SEQ ID NO: 3 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagcgctggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 4 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHSTYVPGTYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 5 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcactcaacatacgttccgggaacgtatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 6 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYAIYTKVTAFLKWIDRSMKTRGLPKAKSHAPEVITSSPLK

SEQ ID NO: 7 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgcaatctacaccaaggtcaccgccttcctcaagtggatcgacaggtccatgaaaaccaggggcttgcccaaggccaagagccatgccccggaggtcataacgtcctctccattaaag

SEQ ID NO: 8 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWAEGCARKGKYGIYTKVTAFLKWIDRSMKTRGLPKAKSHAPEVITSSPLK

SEQ ID NO: 9 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctgggcggagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 10 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDAAQGDSGGPHVTRFKDTYFVTGIVSWGEGAARKGKYGIYTKVTAFLKWIDRSMKTRGLPKAKSHAPEVITSSPLK

SEQ ID NO: 11 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgccgcgcagggggacagcgggggcccgcacgtcacccgcttcaaggacacctacttcgtgacaggcatcgtcagctggggagagggcgcggcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 12 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHKNYQPDTYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 13 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcacaagaattaccagccagacacttatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagctggggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag

SEQ ID NO: 14 - protein, 450 aa, synthetic construct
GPANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHSTYVPGTYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSAGEGCARKGKYGIYTKVTAFLKWIDRSMKTRGLPKAKSHAPEVITSSPLK

SEQ ID NO: 15 - DNA, 1350 bp, synthetic construct
  source 1..1350: mol_type = other DNA
gggcccgccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattattcacacggaagctctgcagcctggacaacggggactgtgaccagttctgccacgaggaacagaactctgtggtgtgctcctgcgcccgcgggtacaccctggctgacaacggcaaggcctgcattcccacagggccctacccctgtgggaaacagaccctggaacgcaggaagaggtcagtggcccaggccaccagcagcagcggggaggcccctgacagcatcacatggaagccatatgatgcagccgacctggaccccaccgagaaccccttcgacctgcttgacttcaaccagacgcagcctgagaggggcgacaacaacctcaccaggatcgtgggaggccaggaatgcaaggacggggagtgtccctggcaggccctgctcatcaatgaggaaaacgagggtttctgtggtggaaccattctgagcgagttctacatcctaacggcagcccactgtctctaccaagccaagagattcaaggtgagggtaggggaccggaacacggagcaggaggagggcggtgaggcggtgcacgaggtggaggtggtcatcaagcactcaacatacgttccgggaacgtatgacttcgacatcgccgtgctccggctcaagacccccatcaccttccgcatgaacgtggcgcctgcctgcctccccgagcgtgactgggccgagtccacgctgatgacgcagaagacggggattgtgagcggcttcgggcgcacccacgagaagggccggcagtccaccaggctcaagatgctggaggtgccctacgtggaccgcaacagctgcaagctgtccagcagcttcatcatcacccagaacatgttctgtgccggctacgacaccaagcaggaggatgcctgccagggggacagcgggggcccgcacgtcacccgcttcaaggacacctacttcgtgacaggcatcgtcagcgctggagagggctgtgcccgtaaggggaagtacgggatctacaccaaggtcaccgccttcctcaagtggatcgacaggtccatgaaaaccaggggcttgcccaaggccaagagccatgccccggaggtcataacgtcctctccattaaag